(19) United States
(12) Patent Application Publication
(10) Pub. No.: US 2005/0182402 A1
(43) Pub. Date: Aug. 18, 2005

(54) Device at medullary nails for fixation of bone fragments at bone fractures

(76) Inventor: Henrik Hansson, Vreta Kloster (SE)

Correspondence Address: Tarolli Sundheim;Covell & Tummino, Suite 1111, 526 Superior Avenue, Cleveland, OH, 44114-1400, US

(21) Appl. No.: 10/509,609

(22) PCT Filed: Apr. 2, 2003
(86) PCT No.: PCT/SE03/00524

(30) Foreign Application Priority Data
Apr. 4, 2002 (SE) 0201011-4

(51) Int. Cl. A61B 17/72

(52) U.S. Cl. 606/64; 606/68

(57) ABSTRACT

The present invention relates to a device at medullary nails for fixation of bone fragments at bone fractures, wherein a medullary nail (5) is insertable into holes (6) in medullary canals in the bone fragments. Front parts (5b) of the medullary nail (5) can be locked to one of the bone fragments by means of a locking means (9) which is transversely located in the bone fragment and extends through a transverse hole (10) in said front parts (5b) of the medullary nail (5). The front members (5b) of the medullary nail (5) includes a snap-in device (11) for attaching with a snap-in action the medullary nail (5) to the locking means (9).

The present invention relates to a device at medullary nails for fixation of bone fragments at bone fractures, wherein a medullary nail is insertable into holes in medullary canals in the bone fragments, wherein front parts of the medullary nail can be locked to one of the bone fragments by means of a locking means which is transversely located in the bone fragment and extends through a transverse hole in said front parts of the medullary nail.

In order to fix the front members of a medullary nail at a bone fragment, one can use a locking means e.g. in the form of a screw which is screwed into a predrilled, transverse hole in the bone fragment and through a transverse hole in said front parts of the medullary nail.

When screwing the screw into the bone fragment, said screw often ends up obliquely relative to the transverse hole in the medullary nail, and for being able also in such cases to screw said screw through said hole, said hole must have a substantially larger or at least a great deal larger diameter than the screw. This means however, that there will be a play between the medullary nail and the screw so that the medullary nail can move particularly in torsional directions but also in axial directions relative to the screw. This in turn, means that the bone fragments will be able to move relative to each other, i.e. the fixation will not be stable.

It is also very difficult to hit the hole in the medullary nail with a drill by means of which the transverse hole is drilled. This is done during radioscopy and it is very common that the surgeon is forced to drill several times on different locations, which results in a strongly weakened bone, while simultaneously the surgeon and the patient are subjected to radiation for an unsuitable long time.

On ulna, radius and fibula, it is not possible to use conventional medullary nails and securing said nails with transverse screws, because the medullary canal in these bones is too small for being able to use medullary nails having sufficiently large diameter for providing them with the required large hole for a transverse screw.

The object of the present invention is to remedy, these problems and accomplish that the medullary nail can be fixed substantially immovable at the locking means. This is arrived at by providing the device according to the invention with the characterizing features of subsequent claim 1.

Since the medullary nail has a snap-in device, allowing said nail to be snapped-in onto the locking means, it is accomplished that the front members or parts of the medullary nail can be fixed substantially immovably at the locking means. Furthermore, the snap-in device can be designed such that the locking means easily captures the medullary nail when said nail is threaded into the medullary canal.

The invention will be further described below with reference to the accompanying drawing, in which

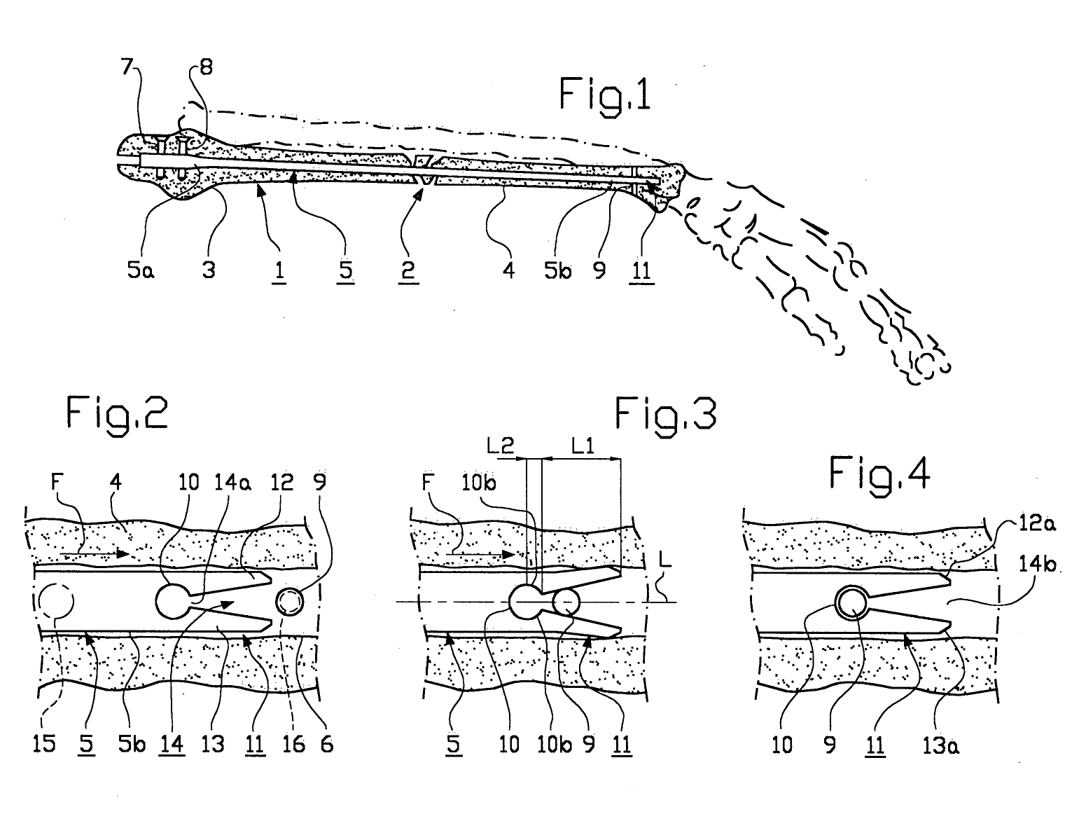

FIG. 1 is a longitudinal vertical section of a forearm bone with a medullary nail according to the invention; and

FIGS. 2, 3 and 4 illustrate a longitudinal horizontal section of front parts of the medullary nail of FIG. 1 in different positions relative to a locking means during a fixation movement at which the medullary nail is fixed at the locking means.

The forearm bone 1 (ulna) illustrated with a section in FIG. 1 has a bone fracture 2 at which the forearm bone 1 is broken in two or more bone fragments, e.g. an upper bone fragment 3 and a lower bone fragment 4.

These bone fragments 3, 4 are fixed relative to each other by means of a medullary nail 5 which is inserted in a hole 6 which has been drilled in the longitudinal direction in the medullary canals of the bone fragments 3, 4. In the upper bone fragment 3, a number of holes, e.g. two transverse holes, have been drilled for two transverse locking screws 7, 8 or corresponding locking means which extend through two transverse holes in rear parts 5a of the medullary nail 5. The transverse locking screws 7, 8 are adapted to fix the upper bone fragment 3 and the rear parts 5a of the medullary nail 5 relative to each other. In the lower bone fragment 4 a transverse hole has been drilled into which a transverse locking screw 9 is screwed. This locking screw 9 is adapted to extend through a transverse hole 10 in front parts 5b of the medullary nail 5. The locking screw 9 is adapted to fix the lower bone fragment 4 and the front parts 5b of the medullary nail 5 relative to each other and it can be extracted from the hole 10 by being backed out therefrom and from the hole therefor in the lower bone fragment 4.

The instrument for drilling a longitudinal hole in the medullary canal for medullary nails 5 and transverse holes for locking screws, are commonly known and therefor not further described.

The front parts 5b of the medullary nail 5 has a snap-in device 11 for attaching by snap-in action said front parts 5b to the transverse locking screw 9. The snap-in device 11 is designed to be threaded onto the locking screw 9 by moving it in a forward direction in the hole 6 in the medullary canal as is shown with an arrow F in FIGS. 2 and 3. Hereby, the snap-in device 11 is brought to be opened by the locking screw 9—as is apparent from FIG. 3—and when the locking screw 9 is situated in the hole 10, the snap-in device 11 will snap-in to its closed condition—as is shown in FIG. 4—which means that the snap-in device 11 retains the medullary nail 5 at the locking screw 9.

The snap-in-device 11 is preferably designed such that it can hold the medullary nail 5 at the locking screw 9 with a firm grip such that the medullary nail 5 can move neither in axial direction relative to its longitudinal axis L nor rotate relative thereto.

The snap-in device 11 is also preferably designed such that it can not loosen or be drawn away from the locking screw 9.

The snap-in device 11 can be designed in different ways for obtaining the abovementioned snap-in result at the locking screw 9. The snap-in device 11 illustrated in the drawings has two jaws 12, 13 which define between them a gap 14 which is open in forward direction and of which inner parts 14a are open towards the hole 10. Said inner parts 14a of the gap 14 has a smaller width than the hole 10 and the diameter of the locking screw 9. The jaws 12, 13 are elastic such that the locking screw 9 brings them to spring in a direction from each other when the snap-in device 11 is threaded thereon. When the snap-in device 11 has been threaded so far onto the locking screw 9 that said screw is situated in the hole 10, the jaws 12, 13 have sprung back to their starting positions due to their elasticity, whereby said jaws 12, 13 retain the medullary nail 5 on the locking screw 9.

The jaws 12, 13 can be provided such that the gap 14 tapers successively in a direction towards the inner parts 14a thereof and to the hole 10. The jaws may further be provided such that outer parts 14b of the gap 14 are rather much wider than the diameter of the locking screw 9, which means that the snap-in device 11 is easier oriented (if necessary by rotating the medullary nail 5 somewhat about its longitudinal axis L) relative to the locking screw 9 when said device shall be threaded onto said screw.

In the embodiment shown, the snap-in device 11 has a keyhole like shape defined by the gap 14 between the jaws 12, 13 and the hole 10. Seen in the longitudinal direction of the medullary nail 5, the jaws 12, 13 have a substantially greater length L1 than the sides 10a, 10b, of the hole 10 closest to the gap 14 and/or the inner sides of the jaws 12, 13 form smaller angles with the geometric longitudinal axis L of the medullary nail 5, than front portions of said sides 10a, 10b. This means that substantially less compressive forces are needed for pressing the medullary nail 5 on the locking screw 9 than for pulling off said medullary nail 5 from said screw. If the inner parts 14a of the gap 14 are substantially narrower than the diameter of the hole 10, it may further be almost impossible to remove the medullary nail 5 from the locking screw 9 unless exceptionally large pulling forces are used.

The locking screw 9 can be replaced by another locking means. The medullary nail 5 may in a manner known per se consist of a metallic material and may further be designed in a known manner. The snap-in device 11 of the medullary nail 5 may have jaws 12, 13 with bevelled outer edge portions 12a, 13a.

The medullary nail 5 may be designed for use at fractures on other bones that an fractures on forearm bones 1. Thus, the medullary nail 5 may e.g. be designed for use at all fractures on tube bones, e.g. radius, fibula, fibia, femur or humerus.

The front parts 5b of the medullary nail 5 may eventually have a second transverse hole 15 behind the hole 10 (shown with broken lines in FIG. 2). The locking screw 9 may have a hole 16 (shown with broken lines in FIG. 2) or another attachment member for attaching a fixture (not shown) to the locking screw 9. This fixture can be adapted to guide a drill for drilling a second hole for a second transverse locking screw (not shown), which shall be screwed through the hole 15.

The medullary nail 5 may eventually be cannulated for threading onto a guide applied in the bone.

1. Device at medullary nails for fixation of bone fragments at bone fractures,
wherein a medullary nail (5) is insertable into holes (6) in medullary canals in the bone fragments (3, 4), and
wherein front parts (5b) of the medullary nail (5) can be locked to one of the bone fragments (3, 4) by means of a locking means (9) which is transversely located in the bone fragment and extends through a transverse hole (10) in said front parts (5b) of the medullary nail (5),
characterized
that the front parts (5b) of the medullary nail (5) includes a snap-in device (11) which is designed to be threaded onto the locking means (9) and thereby opened by the locking means (9) and to be snapped-in again to a closed condition (FIG. 4) when the locking means (9) is located in the transverse hole (10) in the front parts (5b) of the medullary nail (5) such that the snap-in device (11) retains the medullary nail (5) at the locking means (9).

2. Device according to claim 1, characterized in that the snap-in device (11) is designed to retain the medullary nail (5) in a firm grip at the locking means (9).

3. Device according to claim 2, characterized in that the snap-in device (11) is designed to retain the medullary nail (5) at the locking means (9) such that the medullary nail (5) can move neither substantially in axial direction relative to the longitudinal axis thereof nor rotate in relation thereto.

4. Device according to claim 1, characterized in that the snap-in device (11) is designed such that the medullary nail (5) can not loosen or being pulled off from the locking means (9).

5. Device according to claim 1, characterized in that the snap-in device (11) has two jaws (12, 13) which are provided such that the locking means (9), when the medullary nail (5) is moved in a direction (F) in parallel with its longitudinal axis (L) towards said locking means (9), is located between said jaws (12, 13), eventually after rotation of the medullary nail (5) about said longitudinal axis (L) if necessary.

6. Device according to claim 1, characterized in
that the snap-in device (11) has two jaws (12, 13) defining a gap (14) of which inner parts (14a) are open towards the transverse hole (10) in the front parts (5b) of the medullary nail (5),
that the inner parts (14a) of the gap (14) have a less width than the diameter of the transverse hole (10) and a less width than the width or diameter of the locking means (9),
that the jaws (12, 13) are elastic such that the locking means (9) brings said jaws to spring apart when the snap-in device (11) is threaded onto said locking means, and
that the jaws (12, 13) due to their elasticity can spring back to their original positions when the snap-in device (11) has been threaded so far onto the locking means (9) that said locking means (9) is situated in the transverse hole (10), whereby the snap-in device (11) retains the medullary nail (5) on the locking means (9).

7. Device according to claim 6, characterized in that the jaws (12, 13) are provided such that the gap (14) tapers in a direction towards its parts (14a) and to the transverse hole (10).

8. Device according to claim 6, characterized in that the jaws (12, 13) are provided such that outer parts (14b) of the gap (14) are wider than the locking means (9).

9. Device according to claim 6, characterized in
that the jaws (12, 13) have a substantially greater length (L1) than sides (10a, 10b) of the hole (10) closest to the gap (14) seen in the longitudinal direction of the medullary nail (5), and/or
that inner sides of the jaws (12, 13) define or form smaller angles with a geometric longitudinal axis (L) of the medullary nail (5) than front portions of sides (10a, 10b) of the hole (10) closest to the gap (14).

10. Device according to claim 6, characterized in that the jaws (12, 13) have bevelled outer edge portions (12a, 13a).

11. Device according to claim 1, characterized in that the locking means (9) is a locking screw which is screwed into a bone fragment (4) and which can be removed or extracted from the transverse hole (10) in the front parts (5b) of the medullary nail (5) by being unscrewed or backed out of the bone fragment (4).

12. Device according to claim 1, characterized in
that the front parts (5b) of the medullary nail (5) has a second transverse hole (15) behind said first transverse hole (10), and
that the transverse locking means (9) has a hole (16) or another attaching member for attachment of a fixture thereto, said fixture being adapted to guide a drill for drilling a second hole in a lower bone fragment (4) for a second transverse locking means which is adapted to extend through the second transverse hole (15) of the medullary nail (5).

13. Device according to claim 1, characterized in that said device is adapted to be used at medullary nails (5) for forearm bones, e.g. ulna.
